(19) United States
(12) Patent Application Publication
(10) Pub. No.: US 2021/0177573 A1
(43) Pub. Date: Jun. 17, 2021

(54) INTRACORNEAL RING SUPPORTED GRAFT AND METHOD FOR CORNEA REGENERATION

(71) Applicants: Khosrow Jadidi, Tehran (IR); Seyed Aliasghar Mosavi, Tehran (IR)

(72) Inventors: Khosrow Jadidi, Tehran (IR); Seyed Aliasghar Mosavi, Tehran (IR)

(21) Appl. No.: 17/073,314

(22) Filed: Oct. 17, 2020

(30) Foreign Application Priority Data
Dec. 12, 2019 (IR) 13985014000300833

(51) Int. Cl. A61F 2/14; A61L 27/36; A61L 27/16

(52) U.S. Cl. CPC A61F 2/147; A61L 27/3604; A61L 27/3641; A61F 2002/0081; A61F 2230/0006; A61F 2230/0065; A61L 27/16

(57) ABSTRACT

An intracorneal ring made of a polymer compatible with corneal metabolism such as polymethyl methacrylate (PMMA) in cooperation with a corneal graft for adjusting the topography and increasing the thickness of cornea. The ring is a full circle with two spherical or aspherical side surfaces defining its thickness, and an inner and an outer rim defining its width. A plurality of holes distributed around the ring pass through the thickness. A donor corneal graft sutured to the ring via the holes and stretched flat by the suture, fills the inside of the ring. The integrated ring, graft, and suture called as ring graft is inserted in the cornea stroma using superior scleral tunnel incision to correct the shape of the cornea and improve visual acuity. The ring supports the graft circumferentially and promotes its bonding with stroma. The supported graft reshapes and regenerates the cornea. The invention is applicable to treating mild to severe keratoconus, ectasia and other cornea disorders. The ring graft also increases the thickness and strength of cornea, which additionally slows down the progress of keratoconus. There is post operation potential for further visual acuity improvement with refractive correction procedures such as customized PRK. The ring has an outer rim diameter of nearly the normal diameter of cornea and acts as an auxiliary limbus providing additional support to the cornea.

This application has priority date of provisional patent application 139850140003008335 filed 12 Dec. 2019 Granted 19 May, 2020 Iran.

### FIELD OF THE INVENTION

This invention is in the field of ophthalmology and relates to regenerating the cornea with applications in ectasia and keratoconus disease, devices, and methods for their treatment. It also relates to other modifications of cornea for correction of myopia and astigmatism and prevention or treatment of different eye disorders as well as cosmetic purposes.

### BACKGROUND OF THE INVENTION

A normal (emmetropic) eye focuses the image of distant objects on the retina at rest without accommodation. Such an eye creates a sharp vision for distant objects without effort. Any abnormality in the eye constitutes ametropia, a condition where the eye at rest is unable to focus the image of a distant object on the retina. The result is blurred vision.

Ametropia is any condition of myopia (nearsightedness), hyperopia (farsightedness), or astigmatism (cylindrical image distortion). It may come from deviations in geometry of the globe or cornea.

Keratoconus is a chronic progressive noninflammatory eye disorder caused by thinning of cornea and its irregular deformation. The cornea assumes a conical shape which induces ametropia, decreasing vision quality. Keratoconus is a common corneal disease, with a prevalence of approximately 1 in 500 to 2000 according to NIH information.

From a biomechanical viewpoint, the corneal tissue is a viscoelastic material that can change structurally and deform under a force such as intraocular pressure. In some cases, biomechanical structural changes related to keratoconus lead to progression of the disease.

Various treatments have been suggested for the management of keratoconus, including corneal collagen crosslinking (CXL), intrastromal corneal ring (ICR) implantation, and lamellar or penetrating keratoplasty.

CXL uses riboflavin drops combined with UVA light to strengthen the cornea and reduce the progress of Keratoconus.

ICR is generally a rigid implant made of a biocompatible material such as polymethyl methacrylate (PMMA). An ICR may be a full 360° circle, or a circular arc with arc lengths of less than 90° up to 355° called ICR segment. Two ICR segments with equal or different size may be used based on the severity and specific topography of the keratoconic eye. Usually, an ICR has one positioning hole at each end.

Lamellar and penetrating keratoplasties are interventions in which partial or total transplantation of the corneal tissue is performed to restore visual acuity.

ICR is primarily implanted through an incision in the cornea. A pocket is created inside corneal stroma to receive the ICR implant. The pockets may be created with mechanical devices such as PocketMaker microkeratome and Melles hook, or with femtosecond laser. Femtosecond laser is the preferred device as it provides much higher precision and is minimally invasive. The implanted ICR in the corneal stroma reduces the curvature of the cornea and improves visual acuity.

Cornea Implant, U.S. Pat. No. 8,092,526 describes a full circle flexible ring that can be squeezed in the ring plane to a smaller width for insertion into a corneal pocket through an incision in the cornea. After insertion, the ring springs back to a full circle inside the pocket. This ring is available in the market under the commercial name Myoring.

Prior art regarding the intrastromal implantation of PMMA rings for treatment of keratoconus and mild myopia is well documented under U.S. Pat. Nos. 4,452,235; 4,671,276; 4,766,895; 4,961,744; and 8,394,140 and others. All the related patents generally disclose implantation of a full circle or isolated ring segments inside the corneal stroma. Intacs, Keraring, and Myoring are typical ICR devices among numerous commercially available versions.

U.S. Pat. No. 6,051,023 describes an open ICR with one hole on one end and a few holes on the other end for adjusting the size and clipping of the ring after insertion into the cornea.

The cross section of existing ICRs is triangular (Ferrara ring, Myoring, Keraring brands), hexagonal (Intacs brand), or oval (Intacs SK brand). The intrusive geometry and sharp edges of the ICR profiles in relation to the cornea may be one reason for corneal melting.

Several studies report the effectiveness of the rings for improving visual acuity and reducing the refractive error and the mean keratometry (K) value in cases of keratoconus. In addition to the benefit of corneal remodeling and improvement in the optical quality of the cornea, some long-term studies report that ICR implantation may reduce the progression of keratoconus.

For example, the inventors of the present invention have shown that implantation of an ICR (355° Keraring) can regularize the corneal shape and reduce astigmatism in keratoconus patients with a clear cornea and contact lens intolerance (Jadidi, Khosrow, et al., Intrastromal corneal ring segment implantation (Keraring) 355° in patients with central keratoconus: 6-month follow-up, Journal of ophthalmology 2015).

The inventors of the present invention have also tried intrastromal corneal graft for treatment of keratoconus patients. The technique uses femtosecond laser to create a desirable corneal lenticule with precise diameter, thickness, and shape as well as an intrastromal pocket (Jadidi, K., & Mosavi, S. A. 2018, International Medical Case Reports Journal, p. 9-15, Keratoconus treatment using femtosecond-assisted intrastromal corneal graft (FAISCG) surgery: a case series).

In another study, Dragnea et al. developed Bowman layer transplantation for patients with advanced, progressive keratoconus aiming at corneal stabilization (Dragnea et al. Eye and Vision 2018 5:24, Bowman layer transplantation in the treatment of keratoconus). The technique consists of transplanting a donor Bowman layer into a mid-stromal pocket of a keratoconic cornea resulting in corneal flattening and stabilization against further ectasia. The treatment seems to halt the progression of keratoconus but has similar limitations in improving the corneal profile and visual acuity for the same reason of lack of tissue support addressed in Jadidi & Mosavi work above.

Soosan Jacob developed corneal allogenic intrastromal ring segments (CAIRS). CAIRS threads semi-circular donor inserts into channels cut in the patient's mid-peripheral stroma to reinforce and reshape the corneal surface. CAIRS may be done with or without CXL (Soosan Jacobs et al., J Refract Surg, 2018 May 1; 34(5):296-303, Corneal Allogenic Intrastromal Ring Segments (CAIRS) Combined With Corneal Cross-linking for Keratoconus).

Analysis of the ineffectiveness of corneal graft alone in treating keratoconus addressed above points to lack of limbal support and visible change in biomechanics of cornea. Stromal collagens are directional and extend end to end to limbus where they are linked to and supported by limbus. Any cross cutting of cornea permanently weakens it. Specifically, the permanent weakening of cornea due to vertical cutting in LASIK procedure is well documented and established. In keratoconus patients, the overall corneal structure is weak. The additional graft layer does not have the limbal support that is naturally available for stromal lamella and is free to readily deform and conform to the existing topography of the cornea. Therefore, it cannot modify the deformed cornea sufficiently to the desired shape. Gradually, the implanted graft bonds with the stroma, and therefore, the cornea becomes thicker but with nearly the same initial topography. The added graft may help reduce the progression of keratoconus rather than treating it.

Transplanting cornea is a solution to keratoconus but is considered as the last resort because it is complex and invasive, with chances of major complications during and after the operation.

An ICR applies radial outward forces at the boundary of the optical zone of the cornea to reshape it and reduce its curvature. These forces must be transferred through the cross section or thickness of the cornea in the form of shear and tensile forces and bending moment. Therefore, the amount of reshaping and refractive correction is limited by the viscoelastic characteristic of the already weak cornea itself. Also, the edge loading dictates the geometry or profile of the reshaped cornea which may not match the desired shape.

Although ICR has shown to provide some improvement in treatment of keratoconus and its progression, still there is need for ways to better reshape the cornea, further improve visual acuity, and halt the progression in keratoconus disorder. ICR devices can be only used for mild to moderate keratoconus. ICR is not a solution for all keratoconus cases, and therefore, development of new devices and methods can provide further help for treating keratoconus on a wider scale.

### OBJECTS OF THE INVENTION

Therefore, the objects of the present invention include:

1. Transforming and enhancing the overall structure of the cornea by emulating its natural biomechanical system for increased corneal regularity and long term stability.

2. Providing distributed force within the cornea in addition to the localized boundary force of an ICR for improved reshaping of the cornea in both the amount and profile.

3. Providing potential for further conventional visual acuity correction and other corneal procedures and treatments.

4. Providing a treatment option for severe keratoconus over corneal transplantation.

5. Reducing the chance of corneal melting observed with existing ICR devices.

6. Providing a minimally invasive approach to deferring the complex, high risk and high maintenance cornea transplantation.

### DESCRIPTION OF THE DRAWINGS

FIG. 1 shows a top view of the ring graft of the invention.

FIG. 2 shows a sectional side view of the ring graft of the invention in relation to section A-A in FIG. 1

FIG. 3 Shows a detailed view B of the sectional side view of the ring graft of FIG. 2

FIG. 4. shows a top view of a donor corneal graft sutured peripherally to the inner opening of the ring graft of the invention.

FIG. 5 shows a sectional side view D-D in relation to FIG. 4 of the donor corneal graft sutured peripherally to the inner opening of the ring graft of the invention.

FIG. 6 shows a sectional side view of the ring graft of the invention with the graft implanted inside the cornea.

FIG. 7 shows corneal keratometry of the eye before and after the procedure of the present invention.

FIG. 8 shows a table of visual acuity before and after the procedure of the present invention.

FIG. 9 shows a slit lamp photograph of the eye, five days after the procedure of the present invention.

FIG. 10 show corneal topography of the eye before the procedure of the present invention.

FIG. 11 shows corneal topography of the eye, 3 months after the procedure of the present invention.

FIG. 12 shows a computed tomography of the cornea of the eye, one month after the procedure of the present invention (Anterior Segment OCT, Casia SS-1000).

### BRIEF DESCRIPTION OF THE INVENTION

The present invention provides novel device and method to achieve all the objects of the invention discussed above. It corrects and slows down the deformation of cornea due to keratoconus or ectasia. Additionally, it allows to further correct refractive errors through conventional methods after the operation. And finally, the invention provides a viable minimally invasive approach to deferring cornea transplantation by regenerating the cornea and further allowing conventional treatments for other cornea related disorders.

The invention uses an ICR made of a polymer compatible with corneal metabolism such as polymethyl methacrylate (PMMA) in cooperation with a corneal graft to treat keratoconus and similar cornea disorders by adjusting the corneal topography and increasing its thickness.

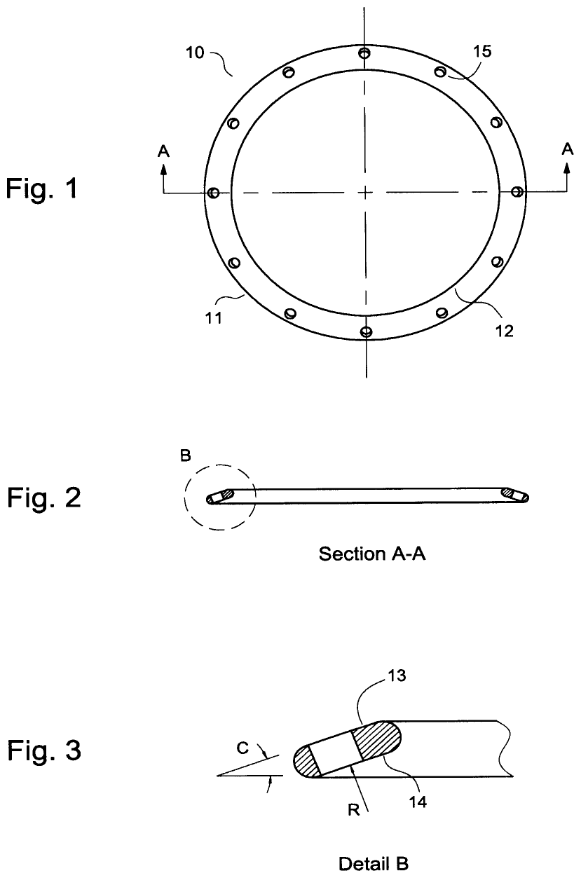

The ring is a full circle with two spherical or aspherical side surfaces defining its thickness, and an inner and an outer rim defining its width. A plurality of holes, preferably 12, distributed around the ring pass through the side surfaces (FIGS. 1, 2, and 3).

The side surfaces of the ring have a curvature in alignment with that of the cornea to prevent corneal melting at the cornea ring interface.

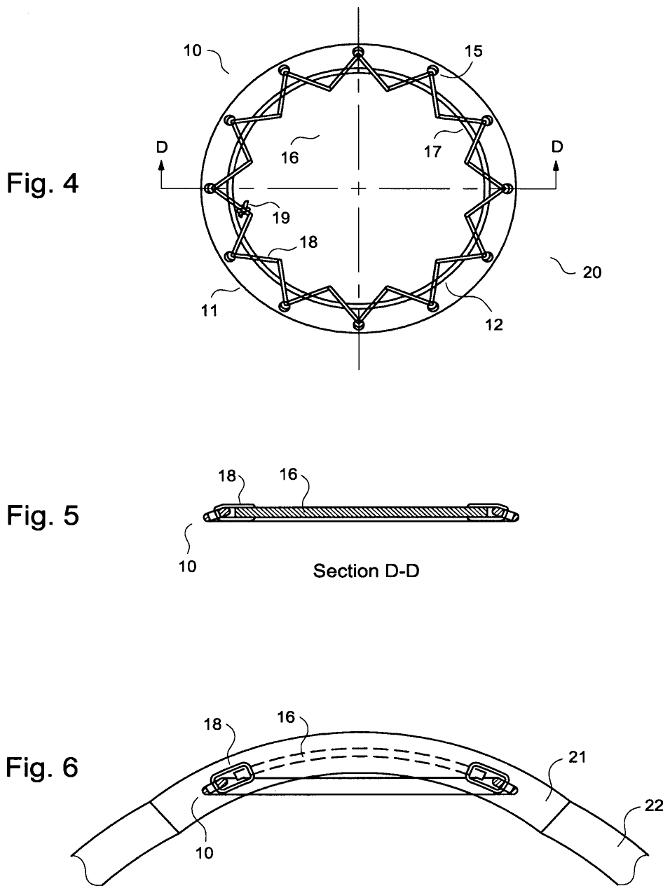

A natural or synthetic corneal graft lenticule is sutured to the ring via the holes such that it is uniformly stretched flat by the suture and fills the inside of the ring. The ring, the suture, and the graft together define a cohesive part acting in unison, named as ring graft in this invention (FIGS. 4 and 5).

The ring graft is inserted in the corneal stroma using superior scleral tunnel incision.

The diameter of ring holes is larger than that of the suture to allow biological interaction between cornea layers on the two sides of the ring and prevent corneal melting.

The cornea tends to align the flat graft with its curvature, which exerts a pull on the ring through the suture. The ring is fixed in location due to its interfacial relation with the cornea. Therefore, due to this interaction between the cornea and ring graft, the cornea stretches and curves the graft, while at the same time the graft reduces the curvature of the cornea (FIG. 6).

This novel way of reducing the curvature of the cornea by the ring graft is in addition to the corneal curvature reduction performed with available conventional ICRs. Besides, it has the advantage of doing so by applying a distributed force within the cornea.

Additionally, the ring exerts an outward radial force that further reduces the curvature of the cornea as a conventional intracorneal ring.

The ring supports the graft circumferentially and promotes its bonding and unification with stroma. The supported graft reshapes and regenerates the cornea. As a major feature of the present invention, this results in increased thickness and strength of the cornea, which advantageously slows down the progress of keratoconus and provides post operation potential for further visual acuity improvement with refractive correction procedures such as customized PRK.

The regeneration of cornea afforded by this invention, provides a minimally invasive approach to deferring the complex, high risk, and high maintenance cornea transplantation. It advantageously opens possibilities for other conventional treatments in cornea disorders.

The ring with its distributed plurality of holes, the cornea aligned side surfaces, the ring graft, the method of preparation of ring graft, or the usage of ring graft in the cornea are exclusive features of the present invention.

### DETAILED DESCRIPTION OF THE INVENTION

The present invention provides novel device and method to achieve all the objects of the invention discussed above. It corrects and slows down the deformation of cornea due to keratoconus or ectasia. Additionally, it allows to further correct refractive errors through conventional methods after the operation. And finally, the invention provides a viable minimally invasive approach to deferring cornea transplantation by regenerating the cornea and further allowing conventional treatments for other cornea related disorders.

The materials, values, and measures in the description of the invention are preferred and typical. They can cover a range of values that is compatible with the biology, geometry, and profile of human cornea.

The descriptions, drawings, and claims of this invention as a whole define and clarify the novel and exclusive features of the invention.

According to FIGS. 1, 2 and 3, there is a circular intraocular ring 10 made of a cornea biocompatible material such as polymethyl methacrylate (PMMA). Ring 10 is a full circle and has outer rim 11, inner rim 12, outer side surface 13, and inner side surface 14. The distance between rims 11 and 12 defines the width, and the distance between side surfaces 13 and 14 defines the thickness of ring 10. There is a plurality of holes 15 distributed around ring 10, passing through the thickness of ring 10 and side surfaces 13 and 14.

The thickness of ring 10 is 0.16 mm. The diameters of outer rim 11 and inner rim 12 are 8.40 mm and 7.60 mm respectively. The diameter of holes 15 is 0.30 mm.

Side surfaces 13 and 14 are preferably spherical or aspherical with a radius of curvature R and an angle C relative to the plane of outer rim 11. Angle C is approximately equal to the cornea angle at the location of ring 10, and therefore, depends on the diameter of outer rim 11, which defines the location of ring 10 relative to the cornea. As the diameter of outer rim 11 becomes smaller, angle C also becomes smaller. The recommended range of value for angle C is 20 to 40 degrees.

From a different perspective, ring 10 is a slice of a spherical or aspherical shell between two parallel planes defined by outer rim 11 and inner rim 12. This feature is intended to prevent the corneal melting issues in existing intracorneal rings.

Holes 15 are diagonally opposite to each other and have a count of 12. Holes 15 allow suturing of a corneal graft to ring 10. Additionally, they facilitate biological interaction and nutrition between cornea layers at the two side surfaces 13 and 14. This feature along with curvature R and angle C prevent corneal melting observed in conventional cornea implants.

Ring 10 with its plurality of distributed holes 15, spherical shell profile, or angle C, is a novel intracorneal ring exclusive to the present invention by itself, in relation to, or in cooperation with a human cornea.

Now, as shown in FIGS. 4 and 5, a circular corneal graft 16 with rim 17 is sutured to ring 10 with suture 18. Graft 16 is either a human or synthetic corneal lenticule, has a diameter of 7.50 mm at periphery 17 to be 0.10 mm smaller than that of inner rim 12, and a thickness of 0.16 mm or equal to that of ring 10. Suture 18 is a 10-0 nylon passing through holes 15 and around periphery 17 of graft 16 successively. The ends of suture 18 are tied together with knot 19.

Suture 18 stretches graft 16 uniformly toward inner rim 12 of ring 10 to a flat circular disk. The diameter of holes 15 is larger than that of suture 18 to allow biological interaction between cornea layers on sides 13 and 14 of ring 10 and prevent corneal melting.

The combination of ring 10, graft 16 and suture 18 work in unison together and define a cohesive and integral unit 20 named ring graft. Ring graft 20 is an exclusive means of the present invention by itself, in relation to, or in cooperation with a human cornea.

Graft 16 is a lenticule cut preferably with femtosecond laser and can be customized for diameter, thickness profile over its area, and shape according to the profile of keratoconus for individual patients. The customized thickness allows rebuilding the cornea to both an increased and uniform thickness across the cornea.

Graft 16 can be pure stroma or include the Bowman's membrane. Use of Bowman's membrane allows benefitting from its high stiffness and using same donor eye for two grafts.

Referring to FIG. 6, ring graft 20 is inserted in the stroma layer of cornea 21 using superior scleral tunnel incision. The insertion method avoids any incision on cornea 21 to maintain its integrity and biomechanics. Ring 10 is positioned at the periphery of cornea 21 and centered with it. Ring 10 can be near limbus 2 or have a smaller diameter at outer rim 11 for implantation closer to the central zone of cornea.

For insertion of ring graft 20 inside the stroma of cornea 21, a circular pocket is created inside the stroma. The pocket is created with Melles hook. Femtosecond laser may also be used instead.

Ring 10 reduces the curvature of cornea 21 by applying outward radial force to it as in existing intracorneal rings. Additionally, ring graft 16 further reduces the curvature of cornea through a separate and more effective mechanism. Ring 10 is fixed in the peripheral zone of cornea 21. Graft 16 is bound outside its normal flat plane by the curved middle zone of cornea 21. Ring 10 pulls graft 16 through suture 18 toward its flat shape while cornea 21 tends to pull graft 16 away and align it with its own curvature. As a result, cornea 21 and graft 16 reach an equilibrium curved state with reduced corneal curvature.

Ring 10 also applies a more effective tangential tension to the periphery 17 of graft 16 in contrast to the mere radial outward force at the ring-cornea interface in existing intracorneal rings.

This novel way of reducing the curvature of cornea 21 by the ring graft 20 is in addition to the corneal curvature reduction performed with available conventional intracorneal rings. Besides, it has the advantage of doing so by applying a distributed force at the interface of graft 16 with cornea 21.

Additionally, ring 10 exerts an outward radial force that further reduces the curvature of cornea 21 as a conventional intracorneal ring.

Ring 10 supports graft 16 circumferentially and promotes its bonding and unification with the stroma of cornea 21. The supported graft 16 reshapes and regenerates cornea 21. As a major feature of the present invention, this results in increased thickness and strength of cornea 21, which advantageously slows down the progress of keratoconus and provides post operation potential for further visual acuity improvement with refractive correction procedures such as customized PRK.

Daxer has shown that a full circle ring implant acts as an artificial limbus and strengthens the cornea (Daxer A., Cornea, p. 1493-1498, Volume 34, Number 11, November 2015, Biomechanics of Corneal Ring Implants). However, existing rings are structurally and isolated form the cornea. In contrast, ring graft 20 integrates and unifies structurally with the cornea, creating a thicker, and therefore, stronger cornea with graft 16, while additionally strengthening the regenerated and enhanced cornea with ring 10 as an auxiliary limbus linked to it through suture 18. In other words, the invention creates a new cornea with additional integrated limbal support.

The tight and stretched condition of graft 16 enhances its binding with the stroma and improves its biomechanical properties. One such property is cornea stiffness measured by a device such as Corvis ST via deformation of cornea under pulses of air. According to clinical data, the invention improved cornea stiffness by a factor of 2 to 3, which is an advantage over existing methods.

Currently, the only proven method of preventing keratoconus progress is CXL. The inventors suggest that the added thickness, strength, and limbal style support of cornea by ring graft 20 of the present invention should provide a definitive way to prevent the progression of keratoconus. The difference comes from the fact that CXL only creates additional links within the existing thin cornea structure.

Corneal irregularity causes glare and halo. Clinical studies by the inventors showed improvement in irregularity and reduction of glare and halo.

According to direct observation by the inventors, all existing ICRs may cause corneal melting. The present invention reduces the chance of corneal melting.

The inventors have also observed that reducing corneal curvature reduces astigmatism. The present invention should improve astigmatism better than existing intracorneal rings.

Currently, for sever keratoconus only transplantation is the norm. The present invention provides treatment for any level of keratoconus. The inventors successfully treated sever keratoconus cases in their clinical studies of the invention.

The regenerating of cornea afforded by this invention provides a minimally invasive approach to deferring the complex, high risk and high maintenance cornea transplantation. It advantageously opens possibilities for other conventional treatments in cornea disorders.

A ring graft stiffness measurement device may be used to adjust the stiffness of graft 16 via suture 18 to apply a desired pressure on cornea 21 for optimal curvature and vision correction of the cornea.

Three parameters, namely, thickness of ring 10, thickness of graft 16, and tension or stiffness of graft 16, can be varied for a desired refractive correction.

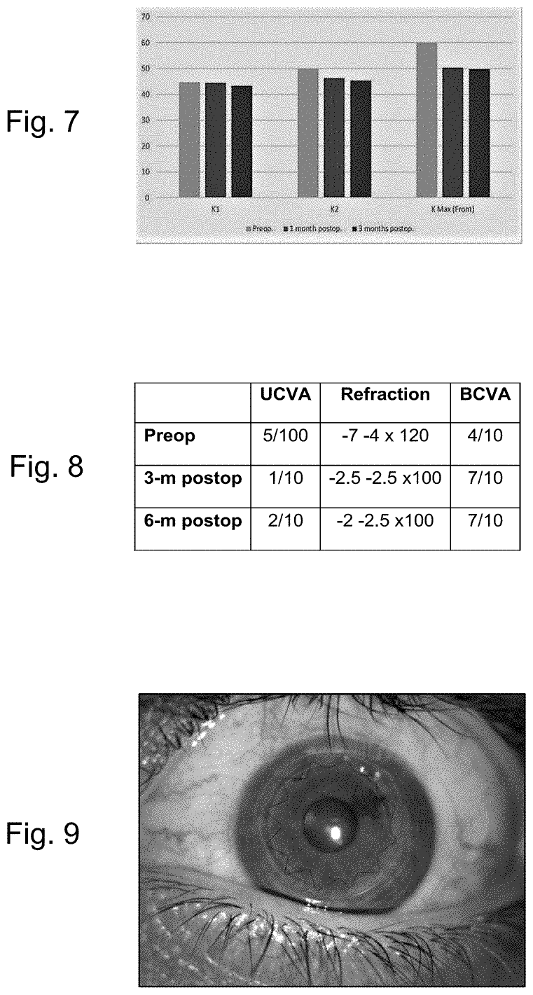

The device and method of the invention were successfully applied in severe keratoconus. The conditions and measurements of one eye are brought here in FIGS. 7 to 12 as a reference and proof of novelty and practicality of the invention.

FIG. 7 shows corneal keratometry of the eye before and after the procedure of the present invention demonstrating decrease in the mean topographic K values. K max reduced from 60 diopter to 50 diopter in three months.

FIG. 8 shows a table of visual acuity before and after the procedure of the present invention indicating significant improvement in uncorrected visual acuity (UCVA) and best corrected visual acuity (BCVA).

FIG. 9 shows a slit lamp photograph of the eye, five days after the procedure of the present invention. The zoomed photograph shows suture 18 in the cornea which is normally invisible to the eye. There is visible regularity with no unusual reaction, the eye is quiet, and the cornea is clear.

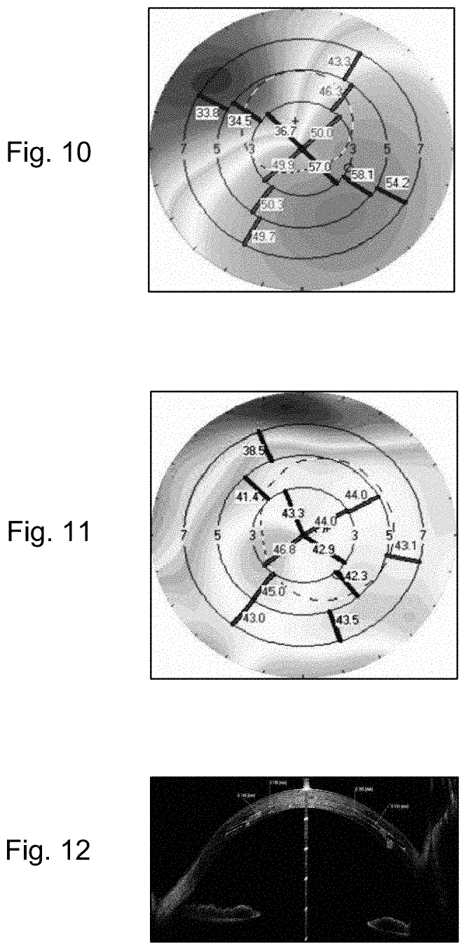

FIG. 10 shows corneal topography of the eye before the procedure of the present invention. The topography shows a severe case of keratoconus. The cornea is totally irregular with cornea inferior steepening and cornea curvature changing from 57.0 diopter to 36.7 diopter in the 3 mm central area of the cornea.

FIG. 11 shows corneal topography of the eye 3 months after the procedure of the present invention. The cornea has become relatively regular. Same preop values of 36.7 and 57.0 are improved to 43.3 and 42.9 respectively. There is high uniformity in the center with values of 43.3, 44.0, 42. 9, and 46.8. The cone that was at the inferior location is moved to the center. Regularity of cornea is restored.

FIG. 12 shows an anterior segment OCT (Casia SS-1000) of the cornea one month after the procedure of the present invention. The graft with marked measurements is in the middle of stroma, has maintained its original 0.16 mm thickness with no shrinkage, is clear and uniform, and unified with the cornea. There is no wrinkling in donor and receiving cornea tissues. There is visible increase in corneal thickness. A new healthy cornea with sufficient thickness and proper shape is created proving the novelty and practicality of the present invention.

The basics of preparing and installing ring graft 20 and the merits of the invention are described in previous paragraphs. The details of the method of the invention will be disclosed below.
1. Create corneal lenticule as graft from donor eye. Preoperatively, remove epitheliums of donor eyes from the whole globe with a blade number 15. Subsequently, create a corneal lenticule with precise diameter and thickness as desired using VICTUS Femtosecond Laser (Bausch+Lomb).
2. Create ring graft 20. Suture graft 16 to ring 10 using 10-0 nylon suture.
3. Create a corneal pocket in recipient's eye. Perform local anesthesia in recipient's eye with tetracaine 0.5% anesthesia droplets (Sina Darou Company) three times in 5 min intervals before, and as needed during operation. Perform peritomy from superior part of cornea 21 and make an incision in the sclera 1.5 mm from limbus. The diameter of incision is about 6 mm. Extend the incision as scleral tunnel into mid stromal part of cornea 21 manually using Melles hook. The pocket is midstromal, 1 mm larger than outer rim diameter 11 (9.4 mm for 8.4 mm ring).
4. Insert ring graft 20 in the corneal pocket. Separate the pocket from the underlying stromal layer with the aid of a Melles hook. Insert ring graft 20 in the cornea. Irrigate pocket with BSS, and stick conjunctiva in place using cauterization without suture.
5. End procedure. Place a silicone-hydrogel bandage contact lens (Alcon Laboratories, Inc., Fort Worth, Tex., USA) on the cornea.
6. Apply post operation regimen. Administer Ciprofloxacin antibiotic 0.3% (Sina Darou Company) one drop every 6 hours for one week. Administer betamethasone (Sina Daroo Company) one drop every 4 hours for one week, then taper for 6 months, plus artificial tear drop or lubricant. Remove the contact lens after 2 days when the wound has healed.

The ring with its distributed plurality of holes or the cornea aligned side surfaces, the ring graft, the method of preparation of ring graft, or the usage of ring graft in the cornea are exclusive to the present invention.

We claim:

1. An intracorneal ring made of a polymer compatible with corneal metabolism such as polymethyl methacrylate (PMMA), to be used in cooperation with a corneal graft for adjusting the cornea topography and increasing its thickness,
the ring is a full circle with two side surfaces defining its thickness, and an inner and an outer rim defining its width,
a plurality of holes distributed around the ring pass through the thickness of the ring.

2. The intracorneal ring of claim 1 wherein the ring is part of a spherical or aspherical shell sliced between two parallel planes.

3. The intracorneal ring of claim 1 wherein the side surfaces of the ring have an angle of 20 to 40 degrees relative to the plane of its rims.

4. The intracorneal ring of claim 1 wherein the holes are evenly distributed around the ring.

5. The intracorneal ring of claim 1 wherein the corneal graft is prepared from a human donor's stroma.

6. The intracorneal ring of claim 1 wherein the corneal graft is prepared from a human donor's Bowman's membrane and stroma.

7. The intracorneal ring of claim 1 wherein the corneal graft is a synthetic tissue.

8. The intracorneal ring of claim 1 wherein:
the graft is a circular lenticule with a diameter smaller than the diameter of the inner rim,
the graft is sutured to the ring and stretched flat inside the ring via the suture passing successively through the ring holes and the graft periphery, and the suture ends are tied together via a knot.

9. The intracorneal ring of claim 8 wherein the parameters and dimensions are around the following preferred values, but may change according to the specific needs of a receiving eye:
number of ring holes 12,
ring hole diameter 0.3 mm,
ring outer rim diameter 8.6 mm,
ring inner diameter 7.6 mm,
ring thickness 0.16 mm,
average curvature of ring side surfaces 7.5 mm,
ring width 0.8 mm,
graft thickness 0.16 mm, the difference between graft diameter and inner rim diameter 0.1 mm, and suture size 10-0.

10. The intracorneal ring of claim 8 wherein the ring and sutured graft are inserted in the cornea stroma of a receiving eye to reduce the curvature, increase the thickness, or correct the refraction of the cornea, while reshaping and regenerating the cornea, and enhancing the biomechanical properties of the cornea.

11. The intracorneal ring of claim 10 wherein:
the cornea holds the ring in a fixed position,
the ring pulls the graft via the suture,
the graft pushes against the cornea as a result of the ring pull and tends to reduce its curvature,
the cornea pushes against the graft and tends to align the graft with its own curvature,
both the cornea and graft assume an equilibrium state at a reduced cornea curvature and refraction, and
the ring acts as an auxiliary limbus, supports the graft circumferentially, and promotes bonding of the graft to and its integration with the stroma layer, regenerates the cornea and enhances its biomechanical properties, creating potential for further refraction correction and other corneal disorder treatments, and
additionally, the ring exerts an outward radial force that further reduces the curvature of the cornea as a conventional intracorneal ring.

12. The intracorneal ring of claim 10 wherein the ring is inserted in the cornea stroma using superior scleral tunnel incision.

13. The intracorneal ring of claim 10 wherein the graft is cut using a femtosecond laser according to a topography to evenly add to the thickness of the cornea in its central zone.

14. The device of claim 10 wherein the amount of thickness of the ring, the thickness of the graft, or the tension of graft or suture is used to effect a desired corneal curvature or refractive correction.

15. The intracorneal ring of claim 10 used to treat any type of ectasia including severe keratoconus, and therefore, deferring the need for cornea transplantation.

16. A cornea implantable means to reduce the curvature, increase the thickness, provide refractive correction, or biomechanical enhancement to the cornea, the cornea implantable means comprising:
An intracorneal ring made of a polymer compatible with corneal metabolism such as polymethyl methacrylate (PMMA),
the intracorneal ring is a full circle with two spherical or aspherical side surfaces defining its thickness, and an inner and an outer rim defining its width,
a plurality of holes distributed around the ring passing through the thickness of the ring,
a natural or synthetic circular corneal graft sutured to the ring via the holes and a suture such as nylon 10-0, stretched flat by the suture, extending inside the inner part of the ring.

17. The cornea implantable means of claim 16 as inserted inside the cornea stroma, wherein the ring is positioned in the peripheral zone of cornea and the graft extends in the central zone of the cornea,
the cornea holds the ring in a fixed position,
the ring pulls the graft via the suture,
the graft pushes against the cornea as a result of the ring pull and tends to reduce its curvature,
the cornea pushes against the graft and tends to align the graft with its own curvature,
both the cornea and graft assume an equilibrium state at a reduced cornea curvature with improved refraction,
the ring acts as an auxiliary limbus, supports the graft circumferentially, and promotes bonding of the graft to and its integration with the stroma layer, regenerates the cornea and enhances its biomechanical properties, creating potential for further refraction correction and other cornea disorder treatments, and
additionally, the ring exerts an outward radial force that further reduces the curvature of the cornea as a conventional intracorneal ring.

18. A device and method to reduce the curvature, increase the thickness, provide refractive correction, or enhance biomechanical properties of the cornea, said device and method comprising:
an intracorneal ring made of a polymer compatible with corneal metabolism such as polymethyl methacrylate (PMMA),
the ring is a full circle with two spherical or aspherical side surfaces defining its thickness, and an inner and an outer rim defining its width,
a plurality of holes distributed around the ring pass through the thickness of the ring,
A corneal lenticule is created as graft from a donor eye or a synthetic tissue:
preoperatively, epitheliums of donor eye are removed from the whole globe with a blade such as blade number 15,
subsequently, a circular corneal lenticule is created with diameter, depth, and topography as desired using a device such as VICTUS Femtosecond Laser, the graft has a diameter preferably 0.1 mm smaller than the diameter of the inner rim,
the graft is sutured to the ring to create a ring-graft unit:
the graft is sutured to the ring with a suture such as 10-0 nylon, and stretched flat via the suture passing successively through the ring holes and the graft periphery, the suture ends are tied together via a knot,
a corneal pocket is created in recipient's eye:
local anesthesia is performed in recipient's eye with anesthesia droplets such as tetracaine, three times in 5 min intervals before, and as needed, during operation, peritomy is performed from superior part of the cornea and an incision is made in the sclera with a diameter of about 6 mm at a position of about 1.5 mm from limbus,
the incision is extended as scleral tunnel into mid stromal part of the cornea manually using a tool such as Melles hook or femtosecond laser,
the pocket is midstromal, about 1 mm larger than the outer rim diameter of the ring,
the ring-graft unit is inserted in the cornea pocket:
the pocket is separated from the underlying stromal layer with the aid of a tool such as Melles hook,
the ring-graft unit is inserted in the cornea,
the pocket is irrigated with BSS,
the conjunctiva is stuck in place using cauterization without suture,
the procedure is ended:
a silicone-hydrogel bandage contact lens is placed on the cornea.
post operation regimen is applied:
an antibiotic such as ciprofloxacin, is administered at one drop every 6 hours for one week, betamethasone or its equivalent is administered at one drop every 4 hours for one week, then tapered for 6 months,
artificial tear drop or lubricant is administered,
the hydrogel contact lens is removed after 2 days when the wound has healed.

19. The device of claim 18 wherein the graft lenticule includes the Bowman's membrane.

20. The device of claim 18 wherein the amount of the thickness of the ring, the thickness of the graft, or the tension of graft or suture is used to effect a desired corneal curvature or refractive correction.
